(19) United States
(12) Patent Application Publication
(10) Pub. No.: US 2015/0235539 A1
(43) Pub. Date: Aug. 20, 2015

(54) MULTI-FUNCTIONAL DEVICE HAVING AT LEAST THE ABILITY TO DETECT THE PRESENCE OF A SUBSTANCE

(71) Applicant: Etón Corporation, Palo Alto, CA (US)

(72) Inventors: Skip Thomas Orvis, Murphys, CA (US); Esmail Amid-Hozour, Woodside, CA (US); Sharon Louise Cook, San Francisco, CA (US); Hamid Bagh Shomali, Lafayette, CA (US)

(21) Appl. No.: 14/625,328

(22) Filed: Feb. 18, 2015

Related U.S. Application Data
(60) Provisional application No. 61/941,353, filed on Feb. 18, 2014.

(51) Int. Cl. G08B 21/02; G01N 33/00

(52) U.S. Cl. CPC G08B 21/02; G01N 33/004; G01N 33/0065

(57) ABSTRACT

A device, according to one embodiment, includes at least one visual indicator indicating a status of the device; a first light source; a second light source; a photo detector; and a substance detector configured to activate the first light source in response to detecting at least one substance. Other systems and methods are described in additional embodiments.

### RELATED APPLICATIONS

This application claims priority to U.S. Provisional Appl. No. 61/941,353 filed Feb. 18, 2014, which is herein incorporated by reference.

### FIELD OF THE INVENTION

The present invention relates to a multi-functional device, and more particularly, this invention relates to a multi-functional device having at least the ability to detect the presence of a substance such as carbon monoxide.

### BACKGROUND

Carbon monoxide (CO) is a colorless, odorless, and tasteless gas that is toxic to humans and animals when encountered in higher concentrations. In closed environments, like inside a home, the concentration of CO can easily rise to lethal levels. As a result, products have been developed that are designed to detect the presence of CO gas in order to prevent carbon monoxide poisoning of humans and animals.

However, conventional products are limited in their functionality and essentially have no portability. It follows that conventional products may have limited functionality in a fixed location, but fail to serve any greater purpose to a user.

In sharp contrast, various embodiments described herein are able to function in a wide array of environments detecting at least the presence of one substance, e.g., CO levels in the air. Moreover, other embodiments herein may further incorporate additional features including, but not limited to, a light source as will be described in further detail below.

### SUMMARY

A device, according to one embodiment, includes at least one visual indicator indicating a status of the device; a first light source; a second light source; a photo detector; and a substance detector. Moreover, the substance detector is configured to activate the first light source in response to detecting at least one substance.

A method, according to another embodiment, includes sensing for a presence of one or more substance; entering an alarm condition in response to detecting the at least one substance; activating a light source of a device in response to detecting the one or more substance; and deactivating the light source after a predetermined amount of time and/or in response to detecting a battery charge level below a predefined threshold.

A device, according to another embodiment, includes at least one visual indicator configured to convey a status of the substance detector; a user interface button; a switch; a first light source; a second light source; prongs configured to receive power from a power source; a battery; a timer configured to monitor an amount of use of the device and provide an indication in response to determining the amount of use of the device surpasses an expected life-span of the device; at least one of: a USB port and a pass-through plug; a photo detector; and a substance detector. The substance detector is preferably configured to detect at least one of: carbon monoxide, methane, propane, gas fumes, smoke and radon. Moreover, the substance detector may be configured to activate the first light source and/or enter an alarm condition in response to detecting at least one substance. The battery may be configured to power the device in response to the prongs not receiving power from the power source. The first light is configured to activate in response to the prongs not receiving power from the power source. Furthermore, the photo detector may desirably be configured to activate the second light source in response to detecting light having a brightness below a threshold value, and deactivate the second light source in response to detecting light having a brightness above a threshold value.

Other aspects and advantages of the present invention will become apparent from the following detailed description, which, when taken in conjunction with the drawings, illustrate by way of example the principles of the invention.

### BRIEF DESCRIPTION OF THE DRAWINGS

For a fuller understanding of the nature and advantages of the present invention, as well as the preferred mode of use, reference should be made to the following detailed description read in conjunction with the accompanying drawings.

FIG. 1 is a front view and side view of a device according to one embodiment.

FIG. 2 is a front view of a device coupled to a wall outlet according to one embodiment.

FIG. 3 is a back view and side view of a device according to one embodiment.

FIGS. 4A-4C are perspective views of a method of using a device according to one embodiment.

### DETAILED DESCRIPTION

The following description is made for the purpose of illustrating the general principles of the present invention and is not meant to limit the inventive concepts claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified. It should also be noted that, as used herein, the term “about” with reference to some stated value refers to the stated value ±50% of said value. Further, while illustrative values are presented throughout, this is done by means of example only and are without limitation. Accordingly, any modification, alteration, or equivalent of the various embodiments described herein, as would be appreciated by one having ordinary skill in the art upon reading the descriptions, should also be considered within the scope of this disclosure.

In one general embodiment, a device includes at least one visual indicator indicating a status of the device; a first light source; a second light source; a photo detector; and a substance detector. Moreover, the substance detector is configured to activate the first light source in response to detecting at least one substance.

In another general embodiment, a method includes sensing for a presence of one or more substance; entering an alarm condition in response to detecting the at least one substance; activating a light source of a device in response to detecting the one or more substance; and deactivating the light source after a predetermined amount of time and/or in response to detecting a battery charge level below a predefined threshold.

In yet another general embodiment, a device includes at least one visual indicator configured to convey a status of the substance detector; a user interface button; a switch; a first light source; a second light source; prongs configured to receive power from a power source; a battery; a timer configured to monitor an amount of use of the device and provide an indication in response to determining the amount of use of the device surpasses an expected life-span of the device; at least one of: a USB port and a pass-through plug; a photo detector; and a substance detector. The substance detector is preferably configured to detect at least one of: carbon monoxide, methane, propane, gas fumes, smoke and radon. Moreover, the substance detector may be configured to activate the first light source and/or enter an alarm condition in response to detecting at least one substance. The battery may be configured to power the device in response to the prongs not receiving power from the power source. The first light is configured to activate in response to the prongs not receiving power from the power source. Furthermore, the photo detector may desirably be configured to activate the second light source in response to detecting light having a brightness below a threshold value, and deactivate the second light source in response to detecting light having a brightness above a threshold value.

The following description discloses several preferred embodiments of a multi-functional device and/or related systems and methods. As will soon become apparent, the multi-functional device preferably has at least the ability to detect the presence of a substance such as carbon monoxide.

As previously mentioned, various embodiments described herein are able to function in a wide array of environments detecting the presence of at least one substance, e.g., CO levels in the air. Moreover, other embodiments herein may further incorporate additional features including, but not limited to, a light source. For example, one embodiment may include a portable CO detecting device additionally including a flashlight and/or nightlight.

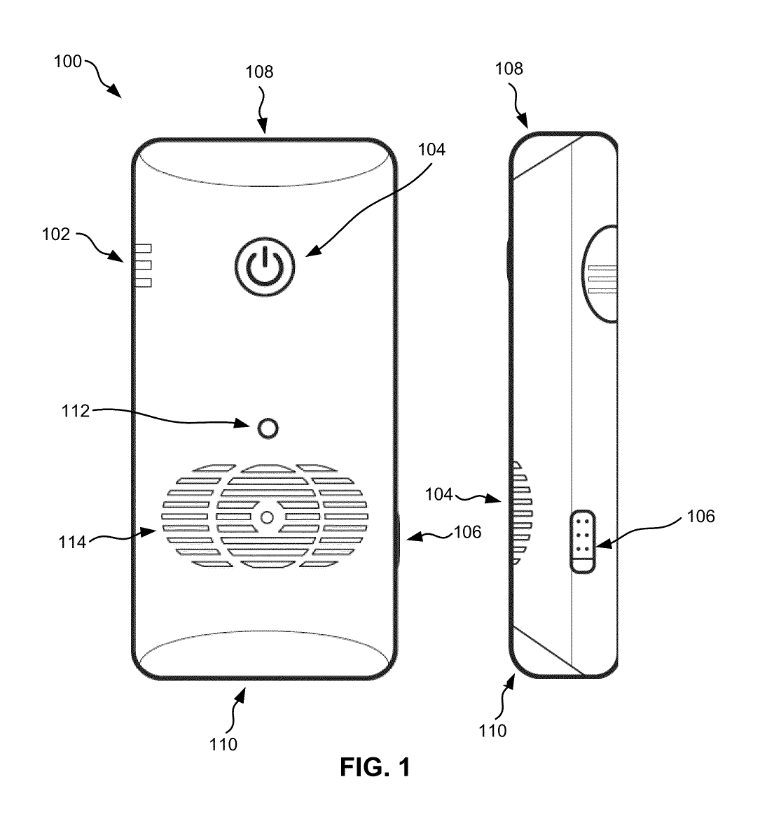

Looking to FIG. 1, a frontal and side profile of an exemplary device 100 are shown. As illustrated, the device 100 includes an array of visual indicators 102, a user interface button 104 (which may act as a power button and/or a multi-function button), a switch 106, a first light source 108 and a second light source 110. The device further includes a photo detector 112 of conventional construction, and a cutout area 114, e.g., allowing audible signals originating from a speaker (not shown) in the device 100 to be emitted.

Unless otherwise specified, any of the components described herein according to any of the approaches may include conventional construction as would be appreciated by one skilled in the art upon reading the present description. As a result, by implementing components of conventional construction, various embodiments herein may achieve improvements in economies of scale in supply of components, reduced approvals compliance risks, and/or increased ease of manufacturability, e.g., using existing production lines.

According to an illustrative embodiment, the speaker (not shown) may provide an audible signal originating therefrom having a sound pressure level of greater than 85 dB at 3 m from the speaker. However, depending on other desired applications, alert conditions, etc., the speaker may emit an audible signal having a different sound pressure level. For example, the speaker may emit audible signals having different pitch, length, rhythm, or speech, etc. depending on the present alert condition.

The device 100 also includes a detector (not shown) for detecting the presence of one or more substances (e.g., also referred to herein as a “substance detector”). In one approach, the detector may be positioned in the device close to the cutout area 114. Thus, the cutout area 114 may serve an additional function of allowing ambient air to be transferred to the detector such that the detector may determine whether one or more substances are present.

In one approach, the detector may be a CO detector. Accordingly, the CO detector may include a CO sensor of a type known in the art. For example, the CO detector may include a CO sensor sold by Figaro USA Inc., having a sales office at 3703 West Lake Ave. Suite 203, Glenview, Ill. 60025 USA. According to an illustrative embodiment, the detector may include an electro-chemical sensor having a sensitivity level at least about the same as the response thresholds depicted in Table 1.

TABLE 1

| | |
|---|---|
| Alarm response threshold^{a} | <30 μL/L no alarm |
| | 79 μL/L within (60-240) min |
| | 150 μL/L within (10-50) min |
| | 400 μL/L within (4-15) min |

Furthermore, the CO detector may also include one or more critical capacitors, potentially having a capacitance of about 1.0 μF, but could be higher or lower depending on the desired embodiment. According to one example, the capacitor may be constructed by Texas Instruments Inc., having a sales office at 12500 TI Boulevard, Dallas, Tex. 75243 USA.

Referring to the device 100 of FIG. 1, the first and second light sources 108, 110 are preferably light-emitting diodes (LEDs). According to various embodiments, the first and/or second light sources 108, 110 may include any conventional LED which would be apparent to one skilled in the art upon reading the present description.

In a preferred embodiment, the first light source 108 includes a single LED having a target current of less than or equal to about 70 mA, but could be higher or lower; and a brightness of greater than about 10 Lumens, but could be higher or lower. However, in another approach, the first light source 108 may include a single LED having an adjustable brightness, e.g., depending on the drive circuit. Further still, in other embodiments, the first light source 108 may include more than a single LED, e.g., two, at least two, three, multiple, etc., depending on the desired embodiment.

In another preferred embodiment, the second light source 110 includes a single LED having a brightness of about 1.8 Lumens, but may be higher or lower depending on the desired embodiment. For example, according to various approaches, the second light source 110 may include an LED having a target current of less than about 20 mA, and a brightness of between about 2.5 Lumens and about 3.5 Lumens, but could be higher or lower. Accordingly, in one approach, the second light source 110 may operate using a current of about 12 mA.

The array of visual indicators 102 preferably include a series of low power LEDs, e.g., having a power consumption less than at least the first and/or second light sources 108, 110. It should be noted that although more than one visual indicator is included in the array of visual indicators 102, in other approaches a device may include one or more (e.g., at least one) visual indicators. Moreover, each of the array of visual indicators 102 may have a different color, e.g., to enable multiple visual signal combinations corresponding to different alarm conditions, as will soon become apparent. In another approach, the second light 110 may be a visual indicator indicating some status of the device, e.g., powered, charging, active, etc.

Moreover, the array of visual indicators 102 may be controlled by an internal processing unit (e.g., a master control unit) to inform a user of a variety of different conditions. Furthermore, the first light source 108, second light source 110, and/or speaker may operate in combination with the array of visual indicators 102 to alert a user. For example, upon detecting the presence of one or more substances, the speaker may emit an audible alarm signal while the first light source 108 and/or the second light source 110 may emit a visual alarm flash to warn a user of the presence of the one or more substances.

It follows that the array of visual indicators 102, first light source 108, second light source 110, and/or speaker may emit a range of different visual and/or audible signal combinations corresponding to each condition that the user may be able to decipher using a table, e.g., see Table 2 below.

TABLE 2

| | Indicator Status | | | | | | |
|---|---|---|---|---|---|---|---|
| Condition | Sounder | Flashlight | Nightlight | Green LED | Red LED | Yellow LED | Pre-requisite |
| Warm up | Off | — | — | Steady on | Steady on | Steady on | |
| | | | | <60 s | <60 s | <60 s | |
| Normal Operating | Off | Off | — | Steady on | Off | Off | |
| condition | | | | | | | |
| CO alarm standby | 1 Beep/45 s | Steady on | — | 1 Flash/45 s | Off | Off | With enough power |
| AC lost | | | | | | | |
| Only power for CO | — | Off | Off | — | — | — | Not enough power |
| detection | | | | | | | |
| Low battery | 1 Beep/45 s | Off | — | Steady on | Off | 1 Flash/45 s | |
| AC INPUT | | | | | | | |
| Low battery | 1 Beep/45 s | Off | Off | 1 Flash/45 s | Off | 1 Flash/45 s | |
| AC lost | | | | | | | |
| CO trouble | 2 Beeps/45 s | — | — | — | — | 1 Flash/45 s | |
| End-of-life | 2 Beeps/45 s | — | — | — | — | 2 Flash/45 s | |
| Alarm | T-4 beeps | flash | Off | — | flash | Off | |
| Test | T-4 beeps | flash | Off | — | flash | Off | |

Looking to Table 2, it should be noted that “Sounder” refers to an exemplary embodiment of the speaker (not shown), “Flashlight” refers to an exemplary embodiment of the first light source 108, “Nightlight” refers to an exemplary embodiment of the second light source 110, and “Green LED”, “Red LED” and “Yellow LED” refer to an exemplary embodiment of the array of visual indicators 102 described above. It should also be noted that “—” indicates that the “Indicator Status” is not influenced by the corresponding “Condition.”

The device may also include a timer to monitor and/or record an amount of use of the device, and alert the user to replace the device as depicted above for the “End-of-life” Condition. Depending on the embodiment, the device may have an expected life-span of about 7 years. Therefore, the timer may monitor and/or record an amount of use of the device, and alert the user to replace the device after about 7 years of use. However, according to other embodiments, the device may alert the user to replace the device should a device's self-test result in any functional component failures. The device may also include a 7 year limited warrantee, e.g., depending on the expected life-span of the device.

While various Conditions are presented above in Table 2, priority levels of each of the Conditions may be assigned to determine a preferred order in which different Conditions are presented, e.g., to a user. A desired order in which the Conditions are presented is as follows: End-of-Life, CO Trouble, Alarm, Low battery, AC Lost.

Although not indicated in Table 2, if an AC power supply for the device is lost (e.g., disconnected) during an alarm or fault condition, the array of visual indicators 102 may only indicate the occurrence by flashing the “Green LED” 1 Flash/45 s.

With continued reference to FIG. 1, the user interface button 104 preferably allows for a user to induce a particular function or functions of the device 100. For example, looking to Table 3, the user interface button 104 preferably complies with the following conditions.

TABLE 3

| | Indicator Status | | | | | | | |
|---|---|---|---|---|---|---|---|---|
| | Function/ | | | | Green | Red | Yellow | |
| Condition | Status | Sounder | Flashlight | Nightlight | LED | LED | LED | Pre-requisite |
| Power Button:* | Hush | Off | Flash once | — | — | flash | Off | Alarm, low battery |
| Short press | | | | | | | | of AC off |
| Power Button: | On/Off Flash | 1 beep when | On/Off | — | — | — | — | Enough power and |
| Short press | LED | operating | | | | | | no alarm |
| Power Button: | No flash LED | 1 beep when | Flash once | — | — | — | — | Not enough power |
| Short press | | operating | | | | | | andno alarm |
| Power Button: | Test | T-4 beeps | flash | Off | — | flash | Off | No alarm or trouble |
| Long press and hold | | | | | | | | |
| Power Button: | Test | — | — | — | — | — | — | Alarm or CO |
| Long press and hold | | | | | | | | Trouble |
| Slide switch: | Auto Nightlight | — | — | >20 lux auto off | — | — | — | |
| Auto | | | | <20 lux auto on | | | | |
| Slide switch: | Nightlight | — | — | On/Off | — | — | — | |
| On/off | On/Off | | | | | | | |

Looking to Table 3, it should be noted that, according to an exemplary embodiment, if the “Sounder” (speaker) is hushed (e.g., turned off) at a time where AC power is not coupled to the device, once the speaker is turned off, it will only reactivate after the device has been recoupled to an adequate AC power supply and then uncoupled from the AC power supply (also referred to herein as the mains supply). Moreover, it should again be noted that “—” indicates that the “Indicator Status” is not influenced by the corresponding “Condition.”

Referring still to Table 3, the “Power Button” (user interface button 104) includes the functionality to turn on and off the “Flash LED” (first light source 108), hush the “Sounder” (speaker), and test the device as a whole. However, according to other embodiments, the user interface button 104 may include additional features. For example, the user interface button 104 may be used to on and off the first light source 108. A single short press of the r interface button 104 may turn on the first Light source 108, while a second short press may turn off the first light source 108.

After being manually activated, the first light source 108 may automatically off after a predetermined amount of time. For example, after being manually activated, the first light source 108 may automatically turn off after 60 minutes. However, the batters level may also determine when the first light source 108 may automatically turn off. For example, when the battery level (as monitored by the device power management unit) reaches a pre-set minimum level, the first light source 108 may automatically turn off.

Furthermore, upon the loss of the mains supply, due to actual AC power failure at an outlet, the device being intentionally or unintentionally unplugged from an AC power outlet, etc., the first light source 108 preferably turns on automatically. However, as mentioned above, after a predetermined amount of time (e.g., 60 min), or when the battery level reaches a pre-set minimum level, the first light source 108 will automatically turn off.

Table 2 additionally illustrates the functionality of switch 106. Preferably the switch 106 has at least an “AUTO” position and an “ON” position. However, referring back to Table 3, the switch 106 has an “AUTO” position, an “OFF” position and an “ON” position. Thus, depending on different embodiments, switch 106 may have added functionality, e.g., corresponding to additional setting positions.

The current consumption of the device preferably does not exceed 50 mA in any given operating mode. It follows that, according to a preferred approach, the device is powered by (e.g., electrically coupled to) a 120 V, 60 Hz AC supply. Ho according to alternate approaches, a device as disclosed herein may be powered by and/or charged by any other voltage and/or current. For example, a device may be powered and/or charged by a 240 V, 50 Hz supply, a 110 V, 60 Hz supply, a 230 V, 50 Hz supply, a 220 V, 50 Hz supply, a 115 V, 60 Hz supply, etc. Thus, devices as disclosed herein may be able to operate and/or charge in situations having different electrical supplies, e.g., thereby gaining worldwide functionality and applicability.

The device further includes a battery, e.g., for continuous operation backup if the AC supply is disconnected. Furthermore, the device may also or alternatively be compatible with an external DC power supply.

The device preferably operates as to ensure that the battery level is maintained in such a way so as to extend the life of the battery. This includes during charging of the battery. For example, device may be able to determine a charge level of the battery therein and adjust the amount of power delivered from the AC supply to charge the battery. Moreover, during charging of the battery, it s preferred that the device remain operational, e.g., a sufficient amount of the power from the AC supply is reserved to ensure operation of the device's components.

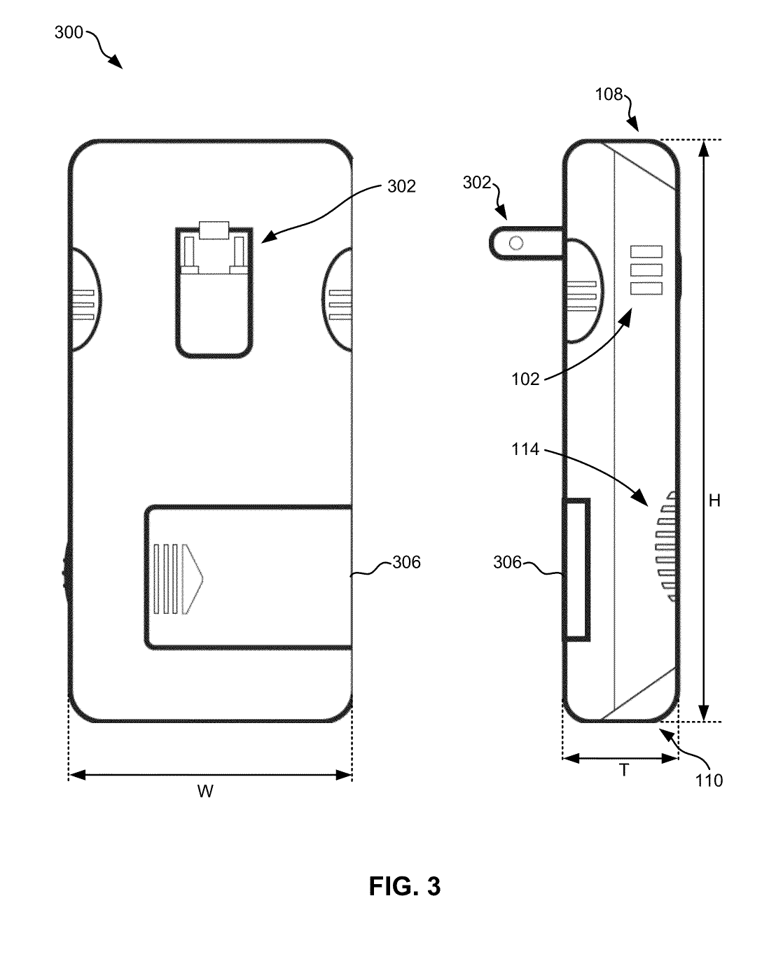

Recognizing that the battery has a finite life span, the design preferably monitors the battery status and alerts a user to replace the battery as necessary to maintain safe operation of the device. Thus, the battery is preferably removable to enable replacement thereof when desired, e.g., upon indication by the device. Therefore, a safe AC supply disconnect method may be provided when the battery is removed for replacement. Furthermore, it is desirable that the device be designed to enable easy access once installed. When the battery is disconnected, the device preferably will have a method to indicate to the user that there is no battery installed. This may be in the form of a light or sound indication once the device is plugged back into AC supply. The devices may also have a mechanical interlock included in the battery compartment such that the battery door (e.g., see 304 of FIG. 3), if so equipped, may not be dosed without installing the battery or defeating the mechanical interlock. Therefore, if a power supply is disconnected, e.g., due to a power outage, the device is unplugged, an electrical breaker is flipped, etc., the device's battery preferably activates about immediately to ensure continued full functionality of the device.

Depending on the battery type, the device may continue to have full functionality for a given period of time. In a preferred embodiment, a device may be able to power the first light source 108 for up to four hours using solely the battery when the battery is fully charged.

According to an exemplary embodiment, the battery may include a replaceable and/or rechargeable battery, preferably having greater than, or equal to about 400 mAh, and a DC voltage of about 3.7 V, but could be higher or lower depending on the desired embodiment. According to different embodiments, the battery may include lithium-ion, nickel-metal hydride, etc. The battery is also preferably a soft pack battery, e.g., to minimize size and weight thereof. However in other approaches, the battery may include a hard pack battery.

In various embodiments, the AC supply power supply may include an electrical outlet. Thus, the device may function while coupled to a fixed power supply (e.g., an electrical outlet) to provide continuous functionality such as substance monitoring and a potential emergency light source.

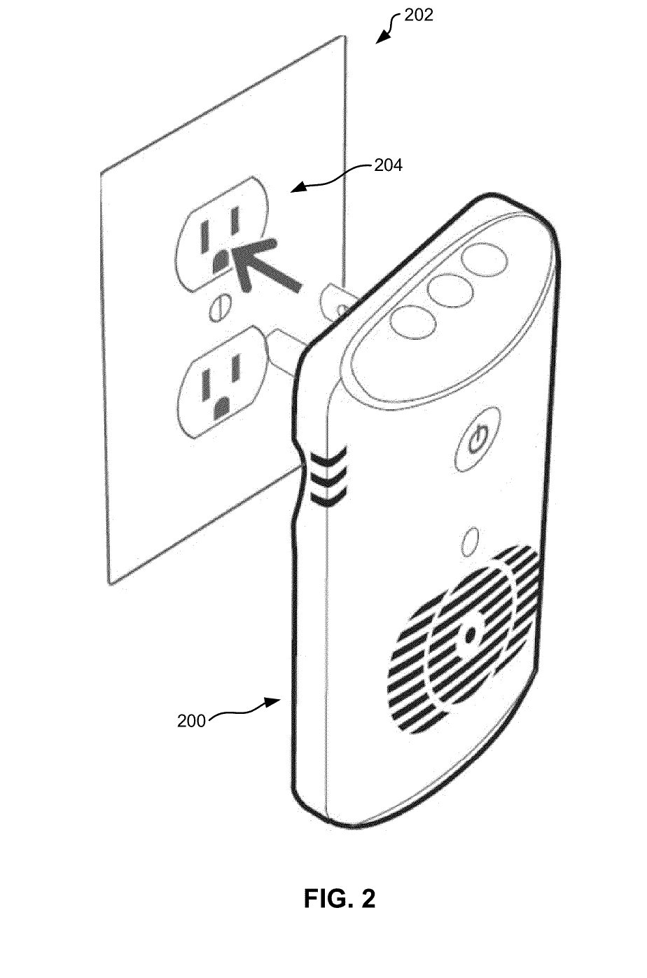

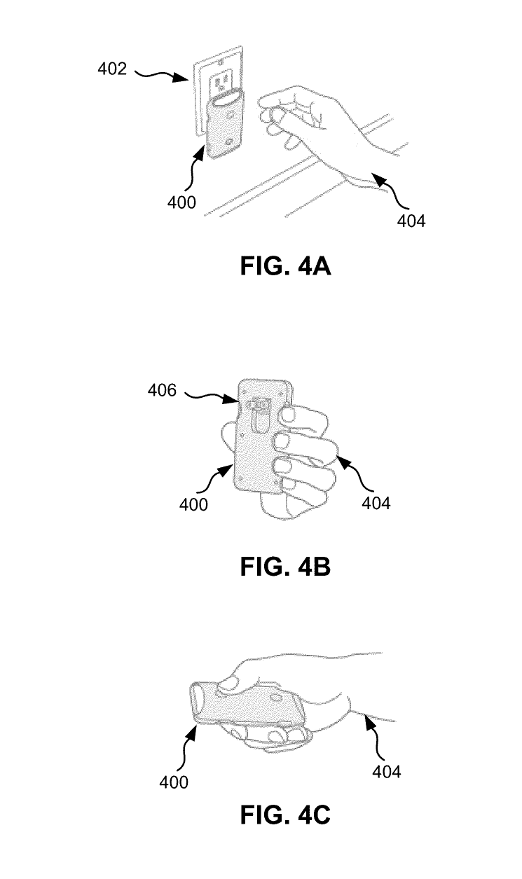

Looking to FIG. 2, when plugged into a first plug an outlet 202, a device 200 may at least partially block access to a second plug 204 of the electrical outlet 202. However, in preferred embodiments, the device 200 does not block access to a second plug of the electrical outlet 202 by positioning the prongs of the device closer to an upper edge of the device, as seen in FIG. 4.

However, referring still to FIG. 2, the device 200 may remain plugged into an electrical outlet for continuous operation and functionality. This may be particularly useful as electrical outlets are usually located near floor level which is a desirable location for CO detection.

Moving now to FIG. 3, a back and side profile of an exemplary device 300 are illustrated according to one embodiment. As an option, the present device 300 may be implemented in conjunction with features from any other embodiment listed herein, such as those described with reference to the other FIGS., such as FIGS. 1-2. Specifically, FIG. 3 illustrates a variation of the embodiment of FIG. 1. Accordingly, various components of FIG. 3 have common numbering with those of FIG. 1.

However, such device 300 and others presented herein may be used in various applications and/or in permutations which may or may not be specifically described in the illustrative embodiments listed herein. Further, the device 300 presented herein may be used in any desired environment. Thus FIG. 3 (and the other FIGS.) should be deemed to include any and all possible permutations.

Looking now to FIG. 3, device includes prongs 302 and battery compartment door 304. Again, the battery (not shown) of the device 300 is preferably removable to enable replacement thereof when desired, e.g., upon indication by the device. The battery compartment door 304 allows for controlled and easy access to the battery compartment, e.g., such that the battery may be removed from the device 300 and replaced with a new battery. The arrow shown on the door 304 in FIG. 3 may indicate the direction in which a force should be applied to the door 304 in order to remove (e.g., detach) the door from the device 300. According to various approaches, the battery compartment door 304 may be coupled to the device 300 using clips, fasteners, friction, adhesives, etc. depending on the desired embodiment.

Moreover, as alluded to above, a device 300 may include foldable prongs 302. Thus, when a user desires to couple the device 300 to an electrical outlet, the prongs 302 may be folded out such that they are in a coupling position. However, when the device 300 is decoupled from an electrical outlet, the prongs 302 may be folded down such that they are in a retracted position as illustrated in FIG. 3. According to different applications, having the prongs folded down to a retracted position may increase the ease of use, increase portability, decrease risk of damage, etc., of the device 300. Additionally and/or alternatively, having the prongs folded down to a refracted position may reduce risk of injury, e.g., for a user.

It should be noted that although the device 300 is depicted as having two prongs 302, a device may have a different number of prongs depending on the desired embodiment. According to an example, which is in no way intended to limit the invention, a device as disclosed herein may include a third prong which serves as a ground plug. However, according to other embodiments, different prong configurations may be implemented, e.g., depending on the geographical region of intended use for a given device. For example, the prong configuration of a device intended for use in North America may be different than the prong configuration of a device intended for use in Europe, as would be appreciated by one skilled in the art upon reading the present description.

An adaptor may be used to enable a device to be compatible with different prong configurations. However, in other embodiments, a device as disclosed herein may include an interchangeable duck head which allows various prong configurations to be interchangeably coupled to the device. According to one approach, an interchangeable duck head may include a pin which may be coupled to a receiving portion of a prong configuration. For example, an interchangeable duck head may allow a two prong configuration intended for use in North America to be detached from a device and a three prong configuration intended for use in Asia to be coupled thereto.

With continued reference to FIG. 3, exemplary dimensions of the device 300 may include a width W of about 2.6 in (6.5 cm), a height H of about 5.1 in (13.0 cm), and a thickness T of about 1.0 in (2.5 cm). Moreover, the device 300 may have a weight of about 4.2 oz (120 g). It should be appreciated that the aforementioned dimensions and weight are in no way intended to limit the invention, and may be higher or lower depending on the desired application.

Looking to FIGS. 4A-4C, an exemplary use embodiment of a device 400 is illustrated. As an option, the present device 400 may be implemented in conjunction with features from any other embodiment listed herein, such as those described with reference to the other FIGS., such as FIG. 4. Of course, however, such device 400 and others presented herein may be used in various applications and/or in permutations which may or may not be specifically described in the illustrative embodiments listed herein. Further, the device 400 presented herein may be used in any desired environment. Thus FIGS. 4A-4C (and the other FIGS.) should be deemed to include any and all possible permutations.

Looking to FIG. 4A, the device 400 is illustrated as being coupled to an electrical outlet 402. However, as a user 404 grabs the device 400 and pulls it away from the electrical outlet 402, the device is decoupled from the outlet 402 and becomes portable as seen in FIG. 4B. Thereafter, the user 404 may fold down the prongs 406 of the device 400 and use the device as a light source for example. See FIG. 4C.

It follows that the device may be used as remote device and/or a removable device even after being unplugged from a power source. The device 400 may have the ability to be completely self-powered and freely positionable in comparison to stationary sensors which require a hard connection (wiring) and have a limited range of placement. Thus, device 400 may be able to continue monitoring while kept stationary in a single location and/or while being transported between different locations, even when not connected to a power source. According to an in-use embodiment, a user may place the device 400 in their pocket to be used as a personal safety device such that the device 400 becomes a sensing alert system having remote monitoring capabilities.

The following example is intended to illustrate an exemplary flow of operation for a device pertaining to one embodiment. Accordingly, the exemplary embodiment is in no way intended to limit the invention. As an option, the present embodiment may be implemented in conjunction with features from any other embodiment listed herein, such as those described with reference to the other FIGS. Of course, however, such embodiment and others presented herein may be used in various applications and/or in permutations which may or may not be specifically described in the illustrative embodiments listed herein. Further, the embodiment presented herein may be used in any desired environment.

According to the exemplary embodiment, a device may be plugged into a power source, e.g., an AC wall outlet. Upon detecting the AC power source, the device may enter into a self-test during which the device tests the battery, photo detector, detector, etc. During the self-test however, the device may be non-functional.

Upon completing the self-test, e.g., after about 60 seconds, the device enters an operation phase in which the device preferably senses for the presence of a substance. Also, during the operation phase, the device preferably charges the battery. As described above, the battery may be charged in such a way as to ensure continued function of the device as a whole.

While the device is sensing for the presence of a substance or substances, if no substances are detected, the second light source (nightlight) may function normally. While the second light source is functioning normally, and when the switch (e.g., see 106 of FIG. 1) is in the OFF position, the second light source remains off. However, when the switch is in the AUTO position, the second light source automatically turns off if the photo detector senses light having a brightness above a threshold value, while the second light source automatically turns on if the photo detector senses light having a brightness below a threshold value.

However, if the device does sense the presence of a substance or substances, the device enters into an alarm state, preferably notifying a user. Referring back to FIG. 1, the array of visual indicators 102, first light source 108, second light source 110, and/or speaker may emit a variety of different visual and/or audible signal combinations as described above. Moreover, the visual and/or audible signal combinations may be deciphered by a user using Table 2 presented above.

Once the device has entered into the alarm state, a user may press the user interface button to clear (Hush) the alarm state. Alternatively, a user may allow the device to remain in an alarm state until the detector no longer senses the presence of the substance, whereby the alarm state is automatically cleared by the device. This instance may be particularly useful in some approaches as the device is not only able to alert a user when a substance is present, but also when the substance has dissipated by automatically clearing the alarm state.

In yet another approach, the AC power source may be disconnected from the device, e.g., from a power outage, a user may physically disconnect the device from the AC power source, etc., whereby the first light source (e.g., see 108 of FIG. 1) is turned on for a predetermined period of time, or for as long as the condition (e.g., charge) of the battery will allow before being depleted below a threshold battery charge level. While the first light source is turned on, and even after the first light source is turned off either automatically after the predetermined period of time, automatically in response to detecting a battery charge level of the battery falls below a predefined threshold charge level, manually by a user, etc., the device remains in an alarm state until a user presses the user interface button to clear the alarm state or the detector no longer senses the presence of the substance, whereby the alarm state is automatically cleared by the device. This instance may be particularly useful in some approaches as the device effectively becomes a hand-held sensing device once a user removes it from an AC power supply.

Alternatively, the AC power source may be disconnected from the device while the device is not in an alarm state. In this situation, the first light source is turned on for a predetermined period of time, or for as long as the condition of the battery will allow before being depleted below a threshold battery charge level. After the predetermined amount of time, or once the battery charge level falls below a threshold, the first light source is automatically turned off. Alternatively, a user may manually turn off the first light source at any desired time.

Even while the first light source is on, the second light source is able to function normally as described above. For example, while the second light source is functioning normally, and when the switch is in the OFF position, the second light source remains off. However, when the switch is in the AUTO position, the second light source automatically turns off if the photo detector senses light having a brightness above a threshold value, while the second light source automatically turns on if the photo detector senses light having a brightness below a threshold value.

If the device remains disconnected from an AC power supply, the device will continue to operate using the charge stored in the battery of the device. However, as the charge of the battery continues to deplete, it will eventually fall below a predetermined “low battery” threshold, whereby the device will indicate to a user that the battery charge level is low.

If the device still remains disconnected from an AC power supply, the device will continue to use the battery of the device until it is completely depleted (to the point where it cannot electrically support the operation of the device), whereby the device will automatically turn off.

However, it should be noted that, at any time during which the device is disconnected from an AC power supply, the device may be reconnected to the AC power supply. Upon detecting the AC power source, the device may enter into a self-test and follow the flow of operations as presented above.

Although various embodiments described herein include detecting the presence of CO, in other embodiments, a detector may be constructed to detect other substances including, but not limited to, methane, propane, gas fumes, smoke, radon, etc. Thus, a device according to any of the approaches described herein may include one or more additional sensing components for detecting one or more additional substances. It follows that a device having additional sensing abilities also maintains multi-functionality as described herein, e.g., having one or more light source, portability, etc.

Furthermore, a device having the ability to detect the presence of more than one substance may also be able to determine which of the more than one substance has been detected and relay that information to a user. For example, a detector may be able to detect the presence of CO, methane, propane, and radon. If the detector detects the presence of radon, it preferably is able to inform a user that radon has been detected, e.g., using a speaker and/or array of visual indicators. Moreover, if the detector detects the presence of radon, and CO, the detector is preferably able to inform a user that both radon and CO have been detected.

According to another illustrative embodiment, a device may include a port to electrically couple the device to an electrical connection. According to one approach, the port may include a Universal Serial Bus (USB) port capable of receiving a cable having a USB connection. Therefore the device may be electrically coupled to another apparatus on the other end of the cable having the USB port. For instance, the cable may connect the device to a computing device which may collect sensing data that may be stored by the device, e.g., corresponding to detected substances. However, in another example, the device may be coupled to an electronic apparatus, such as a mobile phone, whereby the cable supplies power from the battery of the device to charge the battery of the mobile phone.

According to another exemplary use embodiment, the port may be an electrical outlet. Thus, a power cable from another apparatus may be coupled to the electrical outlet of the device. In some approaches, the other apparatus may be powered from the battery of the device, while in other approaches where the device is plugged into an AC power supply (e.g., a wall outlet), the electrical outlet of the device may act as a “pass-through plug.” Referring to the present description, a “pass-through plug” may simply transfer the power from the AC power supply to the power cable of the apparatus, thereby powering the apparatus.

In some embodiments, a device having a pass-through plug may be able to deactivate (e.g., electrically disconnect) the power supply to the apparatus coupled thereto upon detecting a substance. According to an example, which is in no way intended to limit the invention, a device for detecting the presence of flammable vapors, e.g., gasoline, propane, etc., may be coupled to the power cable of a sump-pump in a boat. The sump-pump may be periodically activated, drawing power through the pass-through plug of the device that is plugged into an AC power supply. However, upon detecting the presence of gas vapors, e.g., if the gas tank of the boat begins to leak, the device may deactivate the pass-through plug, thereby cutting power to the sump-pump which would thereby be turned off.

According to further embodiments, a device may be able to induce an action, e.g., deactivate and/or activate a remote power supply, upon detecting a substance. As previously mentioned, a device as disclosed herein may be able to perform one or more actions upon detecting the presence of a substance. According to one approach, a device may be able to send a status signal in response to detecting the presence of a substance. It should be noted that a status signal may include an informational signal, an alert signal, an alarm signal, etc., depending on the situation and desired embodiment. Moreover, upon being received at a target location, a signal sent by the device may induce an action, provide information, trigger an alarm, etc., depending on the desired embodiment.

According to an example, a status signal may be sent by a device to a target location, whereby upon receiving the status signal, a remote outlet at the target location may be deactivated and/or activated. In another example, an informational signal may be sent from a device to a target location, whereby a user, controller, etc. at the target location may react to the informational signal upon receiving it, e.g., by performing an operation, sending a reply signal, processing the signal, etc. The informational signal sent from the device may include monitoring levels of an environmental condition, one or more substances (e.g., gases), etc., over time. It follows that the device may be able to send out nonemergency signals which serve as a stationary monitoring process. Implementations may include the temperature of an area or the presence of a certain amount of gas, e.g., above a threshold. Thus, the device may send an alert, a warning, continuous monitoring information, etc. depending on the desired embodiment. Moreover, depending on the desired approach, a status signal may be sent from a device to a target location and/or user in the form of a telephone call, Multimedia Messaging Service (MMS) message, email, etc.

It follows that devices as disclosed herein may be coupled to a network which enables status signal transmission to a remote and/or local location. In various approaches, the network to which a device is coupled may include a local network, a cellular network, a cloud network, etc., depending on the desired embodiment. Accordingly, a device may be connected to a given network using hardlines, e.g., Ethernet cables, a cellular device, etc. and/or wirelessly, e.g., using any desired wireless standard including, for example, WiFi, Z-Wave, ZigBee, Bluetooth, etc. According to an example, a device may be connected to a local home network by means of a personal mobile device which is connected to the local home network. Accordingly, by paring the device to the personal mobile device, the personal mobile device may serve as a bidirectional gateway to the local home network.

However, in some approaches the device itself may include an internal component (e.g., a cellular device module) coupled thereto which enables a standalone network such as a third generation mobile telecommunications (3G) network, fourth generation mobile telecommunications (4G) network, Long-Term Evolution (LTE) network, etc. According to one example, one or more devices may be interconnected with an existing “mesh” home automation network capable of automating different components, e.g., such as lighting, door locks, etc., thereby forming an interconnected home network solution. Moreover, the one or more devices may be monitored, controlled, in communication with, coupled to, etc. a “mesh” home automation network management device, e.g., a user interface, computing device, automated controller, etc. Thus, a user and/or monitoring system remote from a device may be alerted by sending a signal (e.g., a status signal) using the standalone network. According to an in-use embodiment, an alert signal may be sent to a homeowner and/or a security company from the device directly using a standalone network upon detecting the presence of a substance.

According to another in-use embodiment, a standalone network may be applied to situations in which a user has a vacation home in the mountains. Upon losing power during the winter, a device may send an alert signal (e.g., telephone call, Multimedia Messaging Service (MMS) message, email, etc.) to the user by means of a standalone network maintained by the device in view of its battery backup power supply, regardless of the power outage. The alert signal may inform the user of the power outage, and suggest a manual examination of the water pipes to ensure they are not frozen. However, the alert signal may alternatively or additionally be sent to a local control panel connected to a local network, which may shut off the water in response to receiving the signal information.

In yet another in-use embodiment, a device capable of detecting the presence of a dangerous substance (e.g., CO) may be installed in a home environment and connected to a network via WiFi. Upon detecting the presence of the dangerous substance, the device may send an alert signal to one or more homeowner, security company, neighbor, emergency contact, etc. to inform them. Moreover, upon receiving the alert signal, the receiving entity may take proper action to ensure the safety of any living entities located in the home environment (e.g., pets, children, etc.) regardless of the receiving entity's location.

Embodiments in which one or more devices are implemented in buildings may additionally include a central server connected to each of the one or more devices. Depending on the approach, the central server may be connected to each of the one or more devices wirelessly and/or using hardlines as described above. Accordingly, signals may be sent from the one or more devices to the central server which may perform a local action, e.g., setting off the fire alarm for the building, calling a fire department, etc., depending on the signal received from the one or more devices. According to another approach, a master control panel may be implemented in a home or apartment building which is able to alert members inside the home or apartment building upon the occurrence of an emergency event. Depending on the implementation, the one or more devices may connect to a network which serves as a supervisory network which is based at least on the home or apartment building's already existing network, e.g., the existing network of components that are connected via wires. In other words, the one or more devices may serve as a remote wireless sensor that is also capable of connecting to existing network of components as well.

According to an exemplary in-use embodiment, which is in no way intended to limit the invention, a device may include at least one visual indicator configured to convey a status of the substance detector, a user interface button, a switch, a first light source, a second light source, prongs configured to receive power from a power source, a battery, a timer, a photo detector, a substance detector, a USB port and/or a pass-through plug. The substance detector is preferably configured to detect at least one of: carbon monoxide, methane, propane, gas fumes, smoke and radon, but may be configured to detect the presence of any other desired substance. Moreover, the substance detector may be configured to activate the first light source and/or enter an alarm condition in response to detecting at least one substance. The timer is preferably configured to monitor an amount of use of the device, e.g., compared to an anticipated lifetime of the device, and provide an indication in response to determining the amount of use of the device surpasses an expected life-span of the device. The battery may be configured to power the device in response to the prongs not receiving power from the power source. Thus, the battery may store enough electrical energy to power the device, e.g., until the device is reconnected to a power source, the electrical energy of the battery is depleted, etc. The first light is configured to activate in response to the prongs not receiving power from the power source, e.g., to assist a user in low-light conditions during a power outage, indicate loss of connection to a power source, etc. Furthermore, the photo detector may desirably be configured to activate the second light source in response to detecting light having a brightness below a threshold value, and deactivate the second light source in response to detecting light having a brightness above a threshold value.

It follows that various embodiments described herein preferably provide a user (e.g., a home-owner) with a rechargeable device that provides substance (e.g., CO) detection, and one or more light sources, e.g., a nightflight and/or flashlight, within a single enclosure. The device may function while coupled to a fixed power supply (e.g., an electrical outlet) to provide continuous functionality such as substance monitoring and a potential emergency light source. However, a backup battery will additionally provide the option for continuous environmental monitoring for one or more substance and power the light source in the event that the fixed power supply is unavailable, for instance during portable use of the device. In addition, the device may also provide a nightlight feature that is automatically enabled (with manual override) when ambient light levels falls below a pre-set level.

Moreover, any of the devices described herein may preferably operate under the environmental conditions presented in Table 4 below.

TABLE 4

| | |
|---|---|
| Operating temperature | +4.4° C.~+37.8° C. |
| Operating humidity | 15%~90% RH, non-condensing |
| Storage temperature | −25° C.~+70° C. |
| Storage humidity | 0%~98% RH, non-condensing |

Additionally, various embodiments introduced herein preferably comply with the standards of UL 2034 (including the amendment to change the current surge test to kV), the relevant standards of UL 2075, the additional electrical surge immunity standards specified in UL 217, and with the additional electrical surge immunity standards specified in UL 217 as will be appreciated by one skilled in the art upon reading the present description.

Further still, any of the approaches described and/or suggested herein preferably comply with the design standards presented in Table 5 below.

TABLE 5

| Number | Revision | Title |
|---|---|---|
| IPC-A-610 | E | Acceptability of Electronic Assemblies |

Variations of the device's functionality and/or physical attributes may depend on the region in which sales are intended, region to which they shipping is intended, co-branding strategy, the quantity of devices being shipped, etc.

The inventive concepts disclosed herein have been presented by way of example to illustrate the myriad features thereof in a plurality of illustrative scenarios, embodiments, and/or implementations. It should be appreciated that the concepts generally disclosed are to be considered as modular, and may be implemented in any combination, permutation, or synthesis thereof. In addition, any modification, alteration, or equivalent of the presently disclosed features, functions, and concepts that would be appreciated by a person having ordinary skill in the art upon reading the instant descriptions should also be considered within the scope of this disclosure.

While various embodiments have been described above, it should be understood that they have been presented by way of example only, and not limitation. Thus, the breadth and scope of an embodiment of the present invention should not be limited by any of the above-described exemplary embodiments, but should be defined only in accordance with the following claims and their equivalents.

What is claimed is:

1. A device, comprising:
at least one visual indicator indicating a status of the device;
a first light source;
a second light source;
a photo detector; and
a substance detector,
wherein the substance detector is configured to activate the first light source in response to detecting at least one substance.

2. A device as recited in claim 1, comprising at least one of: a USB port and a pass-through plug.

3. A device as recited in claim 1, wherein the at least one visual indicator is configured to convey a status of the substance detector.

4. A device as recited in claim 1, comprising prongs configured to receive power from a power source, wherein the first light is configured to activate in response to the prongs not receiving power from the power source.

5. A device as recited in claim 1, wherein the substance detector is configured to enter an alarm condition in response to detecting at least one substance.

6. A device as recited in claim 1, comprising a rechargeable battery, wherein the battery is configured to power the device in response to the prongs not receiving power from the power source.

7. A device as recited in claim 1, wherein the photo detector is configured to activate the second light source in response to detecting light having a brightness below a threshold value.

8. A device as recited in claim 1, wherein the photo detector is configured to deactivate the second light source in response to detecting light having a brightness above a threshold value.

9. A device as recited in claim 1, wherein the substance detector is configured to detect at least one of: carbon monoxide, methane, propane, gas fumes, smoke and radon.

10. A device as recited in claim 1, wherein the substance detector is configured to detect carbon monoxide.

11. A method, comprising:
sensing for a presence of one or more substance;
entering an alarm condition in response to detecting the at least one substance;
activating a light source of a device in response to detecting the one or more substance; and
deactivating the light source after a predetermined amount of time and/or in response to detecting a battery charge level below a predefined threshold.

12. A method as recited in claim 11, wherein the one or more substance includes at least one of: carbon monoxide, methane, propane, gas fumes, smoke and radon.

13. A method as recited in claim 11, wherein the one or more substance includes carbon monoxide.

14. A method as recited in claim 11, comprising at least one of the following operations:
performing a self-test of the device; and
charging a rechargeable battery of the device.

15. A method as recited in claim 11, comprising activating the light source in response to the device being disconnected from a power source.

16. A method as recited in claim 11, comprising activating a second light source in response to detecting a light having a brightness below a threshold value; and deactivating the second light source in response to detecting a light having a brightness above a threshold value.

17. A method as recited in claim 11, comprising sending a status signal to a target location in response to detecting the one or more substance.

18. A method as recited in claim 17, wherein the status signal is at least one of: an alarm and monitoring information.

19. A method as recited in claim 17, wherein the status signal is sent to the target location using a network.

20. A device, comprising:
at least one visual indicator;
a user interface button;
a switch;
a first light source;
a second light source;
prongs;
a battery;
a timer;
at least one of: a USB port and a pass-through plug;
a photo detector; and
a substance detector,
wherein the substance detector is configured to activate the first light source in response to detecting at least one substance,
wherein the substance detector is configured to enter an alarm condition in response to detecting at least one substance,
wherein the at least one visual indicator is configured to convey a status of the substance detector,
wherein the timer is configured to monitor an amount of use of the device and provide an indication in response to determining the amount of use of the device surpasses an expected life-span of the device,
wherein the prongs are configured to receive power from a power source,
wherein the battery is configured to power the device in response to the prongs not receiving power from the power source,
wherein the first light is configured to activate in response to the prongs not receiving power from the power source,
wherein the photo detector is configured to activate the second light source in response to detecting light having a brightness below a threshold value,
wherein the photo detector is configured to deactivate the second light source in response to detecting light having a brightness above a threshold value,
wherein the substance detector is configured to detect at least one of: carbon monoxide, methane, propane, gas fumes, smoke and radon.
